(19) United States
(12) Patent Application Publication
(10) Pub. No.: US 2022/0370662 A1
(43) Pub. Date: Nov. 24, 2022

(54) Automated Chemical Injection Device

(71) Applicant: Harold W. Ankrum, Sugar Land, TX (US)

(72) Inventor: Harold W. Ankrum, Sugar Land, TX (US)

(21) Appl. No.: 17/327,473

(22) Filed: May 21, 2021

(51) Int. Cl. A61L 2/18; F24F 13/22; F24F 11/38; A61L 2/24

(52) U.S. Cl. CPC A61L 2/18; F24F 13/222; F24F 11/38; A61L 2/24; F24F 2013/227; A61L 2202/14; A61L 2202/15

(57) ABSTRACT

A system for automizing an injection of a chemical treatment into an effluent drain line may include a containment tank, an injection pump disposed within the containment tank, at least one level switch disposed on or inside the containment tank, a flow line disposed at least partially within the containment tank, at least one flow meter disposed along the flow line, and a microcontroller. The microcontroller is configured to: maintain an off-time cycle, transmit a signal to the injection pump to pump a volume of chemical treatment stored in the containment tank into the flow line after the off-time cycle ends, track the volume of chemical treatment passing through the flow line with the at least one flow meter to produce a flow rate, inject the volume of chemical treatment from the flow line into an effluent drain line, track a current volume of the containment tank with the at least one level, and restart the off-time cycle.

### BACKGROUND

Air conditioners (A/C) are an essential operating system providing comfort to hundreds of millions of Americans. In general, an air conditioner is a device for controlling temperature, humidity, airflows, airflow distribution, and/or the like to allow for human activities to be performed in comfort even through the human endeavor may take place in hot and humid climates. Temperature is controlled and regulated through a refrigeration cycle that is performed and operated by the air conditioner. An air conditioner is a system that may include many elements, such as a compressor, condenser coils, an evaporator, and others which circulate and change the temperature and pressure of a refrigerant as it circulates through the system.

A/C may be classified into many different types of configurations of air conditioners. However, each configuration cools air utilizing an embodiment of evaporator coils. As air is cooled it is also dehydrated, which form condensation. The condensation, or discharge liquid, is collected by a condensation tray. The discharge liquid is collected in a condensation tray and delivered to an effluent drain line indoors to be disposed by plumbing system or taken outdoors. Many times, the discharged water contains organic material that is suspended in the liquid. If left untreated the effluent drain line will become clogged with both organic and inorganic material ultimately forming a thick sludge. To prevent forming of thick sludge A/C manufacturers/service companies recommend homeowners add a small amount of algaecide or chemical cleaning product to the effluent drain line every month to prevent clogging due to sludge (aerobic bacteria, bacteria, mold and organic) buildup. However, this is an often overlooked and possibly dangerous task.

### BRIEF DESCRIPTION OF THE DRAWINGS

These drawings illustrate certain aspects of some examples of the present disclosure and should not be used to limit or define the disclosure.

FIG. 1 illustrates an example of an air conditioning system.

FIG. 2 illustrates a detailed schematic of a split A/C unit.

FIG. 3 illustrates a blockage in an effluent drain line.

FIG. 4 illustrates a schematic of an Automated Chemical Injection Device.

### DETAILED DESCRIPTION

The present disclosure relates generally to methods and systems to treat an effluent drain line and prevent clogging. The injection of chemical treatments prevents buildup of organic materials and thus sludge in the effluent drain line. Sludge may block the effluent drain line, which causes effluent (fluid) to back up in the effluent drain line. This may eventually lead to effluent (fluid) to fill and overflow designated containment units that drain through the effluent drain line. A homeowner may access the effluent drain line and manually inject a chemical treatment into the effluent drain line. However, the system and methods described below for an Automated Chemical Injection Device (ACID) may be implemented to autonomously inject chemical treatment into the effluent drain line. Further, automizing injection of chemical treatment into the effluent drain relieves the homeowner's task to routinely treat the effluent drain line.

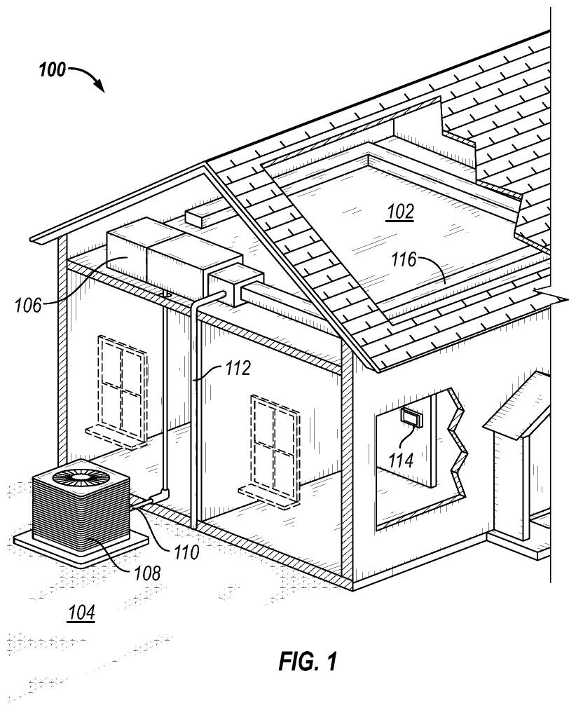

FIG. 1 illustrates an example of a typical air conditioning system 100, which may be defined as a split system. As illustrated, air conditioning system 100 is at least partially disposed in a structure 102 and outside 104. Structure 102 may be any manmade or partially manmade infrastructure such as but not limited to homes, industrial buildings, or apartment complexes. Air conditioning system 100 may include different devices. For example, air condition system 100 may generally include an air handling unit 106, condenser unit 108, refrigerant lines 110, an effluent drain line 112, and a thermostat 114. Thermostat 114 measures the temperature of structure 102 and consistently compares it to a preprogrammed “ideal temperature”. If the temperature of structure 102 is greater than the “ideal temperature” air conditioning system runs as described below until temperature of structure 102 is lower or equal to an “ideal temperature”. In a general setup, air handling unit 106 may be disposed at any suitable location within structure 102. In the illustrated embodiment air handling unit 106 is disposed in an attic but may be disposed anywhere within structure 102. Air handling unit 106 operates and function to cool and dehydrate air, which is distributed through the structure through one or more lines of duct work 116. To cool and dehydrate air, refrigerant is passed through an evaporator coil, discussed below, disposed within the air handling unit 106 in any configuration. Refrigerants employed in standard air conditioning systems 100 are heat carrying mediums which during their cycle absorb heat from a low temperature system and discard it to a higher temperature system. Thus, it is beneficial for refrigerants to have properties such as high critical temperature and low boiling point. Additionally, refrigerants should be non-corrosive to metals.

One or more refrigerant lines 110 pass the refrigerant from air handling unit 106 to condenser unit 108. Refrigerant lines 110 are insulated to maintain refrigerant's temperature inside refrigerant lines 110 and prevent condensation. Further, refrigerant lines 110 are made of copper and prevent leakage of refrigerant between air handler unit 106 and condenser unit 108. Condenser unit 108 chills refrigerant in on or more condenser coils, discussed further below. The chilled refrigerant is transported back to the air handling unit 106 and the evaporator coil using refrigerant lines 110. At this point, hot air is passed over the evaporator coils that are chilled from the chilled refrigerant. The air passing over the evaporator coils is cooled, which also removes water (a dehydration process) from the heated air to create a dry and cool air stream from air conditioning system 100 that is distributed throughout structure 102 by duct work 116. The dehydration process cause water to form in air conditioning system 100, which is captured and drained out of structure 102 through effluent drain line 112. This operation and the functioning of each piece of equipment in air conditioning system 100 is controlled by a thermostat 114.

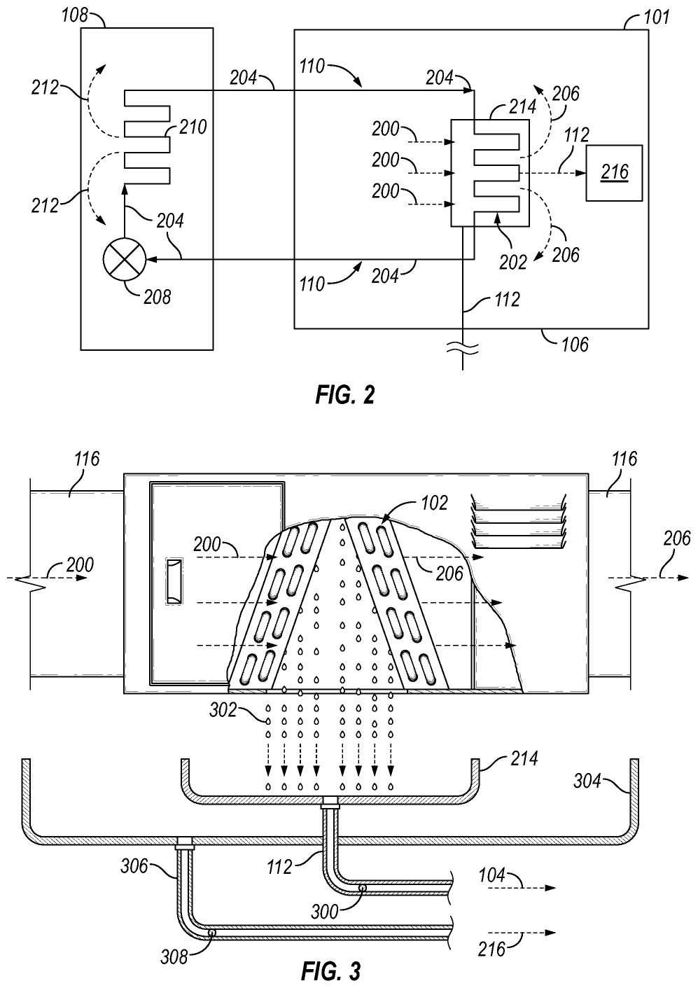

FIG. 2 illustrates a schematic of the flow of air, refrigerant, and temperature through the cycle of a split A/C unit for the process described above. The first step of temperature regulation of a split A/C unit is utilizing hot air 200 disposed in structure 102 (e.g., referring to FIG. 1). In this stage, blower 218 (not illustrated) disposed within duct work 116 (e.g., referring to FIG. 1) blows hot air from structure 102 into air handling unit 106 and across evaporator coils 202. Refrigerant 204 enters the evaporator coils 202 as a cold liquid. Evaporator coil 202 is formed of uninsulated copper tubing aligned in a serpentine configuration. Evaporator coil 202 may be stacked vertically, slanted in an “A shape”, or in any known shape. The configuration of evaporator coil 202 provides a large surface area allowing cool refrigerant 204 to absorb heat. Refrigerant 204 within evaporator coil 202 is not protected by an insulator and transforms into a vapor as hot air 200 runs across evaporator coils 202. Subsequently, hot air 200 loses its heat and is dispersed out of air handling unit 106 and through structure 102 as cool-dry air 206. Additionally, evaporator coil 202 condenses water vapor from hot air 200. In effect, moisture is produced as a byproduct and is allowed to drop into condenser tray 214 as further discussed below.

The vaporized refrigerant is then transferred outside of air handling unit 106 to condenser unit 108. At this stage, refrigerant 204 enters compressor 208, which decreases the vapor's volume and increases its pressure. This creates a difference in pressure and enables the flow of refrigerant 204 through refrigerant lines 110. As refrigerant 204 is compressed by compressor 208 it is compressed into a superheated vapor rather than a liquid. The now superheated refrigerant 204 is transferred to condenser coils 210. A fan 220 (not illustrated) disposed within the condenser unit 108 blows outside air 212 across condenser coils 210. Similar to evaporator coil 202, condenser coil 210 is formed from uninsulated copper tubing aligned in any known configuration, but most commonly a serpentine configuration. As the superheated refrigerant 204 travels through condenser coils 210 its heat is absorbed by the outside air 212. Subsequently, the temperature of refrigerant 204 is lowered and changes from a gas to a liquid. Once the heat from refrigerant 204 is absorbed by outside air 212, the cold refrigerant 204 travels back to air handling unit 106 to evaporator coils 202 to repeat the process described above. The cycle repeats until an ideal temperature is reached within structure 102 (e.g., referring to FIG. 1).

FIG. 3 is an illustrative example of blockage 300, which may form in in effluent drain line 112. As previously mentioned, the by-product of cooling air during the evaporation process at evaporator coils 202 is moisture. Moisture forms as hot air 200 blown in from duct work 116 is dehydrated during the evaporation process, which produces cool-dry air 206 that is also dry and blown out to be dispersed through structure 102 (e.g., referring to FIG. 1) via duct work 116. In effect, discharge liquid 302 may form along evaporator coil 202 due to condensation. A condensation tray 214 is placed directly beneath evaporator coil 202. Thus, when discharge liquid forms, it drips into condensation tray 214. Effluent drain line 112 is connected to air conditioning system 100 through condensation tray 214. Effluent drain line 112 may transport discharge liquid to be disposed of outside 104 (e.g., referring to FIG. 1) or in other embodiments drained into plumbing system 216. During operation, dirt and other debris may collect on evaporator coil 202, and when discharge liquid drips into condensation tray 214 it may take dirt and debris with it. This may result in the formation of sludge in effluent drain line 112. Additionally, due to the location of condensation tray 214, dirt and debris may fall into condensation tray 214. For example, condensation tray 214 may be disposed in an attic in which insulation and other types of debris may float into an open condensation tray 214. In other examples, evaporator coil 202, condensation tray 214, and effluent drain line 112 are moist and damp environments ideal for the growth of algae or mold. In effect, algae or mold may grow and congregate to add to or form a blockage in effluent drain line 112. The air conditioning system 100 described above is in a split system configuration. However, a central system configuration is applicable as well. A central system may also dispose of discharge liquid with effluent drain line 112. Similarly, a blockage may also form in effluent drain line 112 of a central system.

Blockage 300 may be a result of dirt and debris, algae, or mold or a combination of any of the three. Blockage 300 may form a clog and prevent the flow of discharge fluid to outside 104 (e.g., referring to FIG. 1) or to plumbing 216 (e.g., referring to FIG. 2). In effect, discharge liquid 302 may build up and lead to additional costs to be discussed below. In the event of blockage 300 within effluent drain line 112 discharge liquid 302 will overflow into an emergency drain pan 304. In some embodiments, emergency drain line 306 is utilized to transport blocked discharge liquid 302. However even when employed, this a temporary solution as blockage 308 may form as well due to a result of dirt and debris, algae, or mold or a combination of any of the three. In other embodiments, discharge liquid 302 is not transported out of emergency drain pan 304 via emergency drain line 306 and will overfill. Thus, there needs to be a system implemented to remove blockage 300.

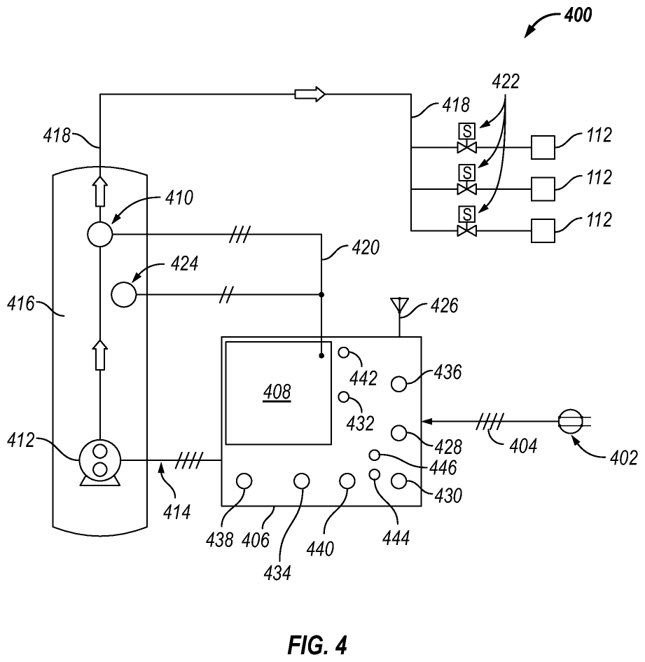

FIG. 4 illustrates an ACID 400, which may be utilized to remove or prevent formation of blockage 300 (e.g., referring to FIG. 3) in effluent drain line 112. In examples, blockage 300 may be sludge, dirt, grime, and/or the like. In embodiments, ACID 400 may autonomize the injection of a chemical treatment into effluent drain line 112. As illustrated ACID 400 utilizing power source 402 which may be connected to the power grid of the house as a typical AC plug. Alternatively, power source 402 may be any standard power supply. Through standard implementation, electrical connection 404 transports 120 VAC to the control panel 406. Disposed on the control panel 406 is microcontroller 408 (not illustrated). In this specific embodiment a power source 402 is used to connect ACID 400 to a power grid, but any other implementation to provide power is suitable as well.

The primary processing and controlling of ACID 400 may be performed by microcontroller 408 as discussed below. Microcontroller 408 transmits control signals via electrical connection 414, electrical connection 420, and internal wiring within the control panel 406. Information traveling in the opposite direction via electrical connection 420 may be processed by microcontroller 408.

The control signal sent via electrical connection 414 activates injection pump 412 disposed within a containment tank 416 to pump an assigned flow volume of chemical treatment into flow line 418. Flow line 418 transports the chemical treatment across flow meter 410 and then out of the containment tank. When ACID 400 is applied to a system with one Air Handler 106 (e.g., referring to FIG. 1) flow line 418 further transports the assigned flow volume to effluent drain line 112. However, in the case of multiple Air Handlers 106 and therefore, multiple effluent drain lines there may be multiple solenoid valves 422 depending on the application of the A/C unit. Each solenoid valve 422 is coupled to effluent drain line 112 injecting the assigned flow volume directly into effluent drain line 112. In an alternative embedment flow meter 410 may be located outside containment tank 416 along flow line 418 before solenoid valve(s) 422.

During operations, microcontroller 408 may transmit a control signal to activate injection pump 412, a timer within microcontroller 408 loaded with an off-time cycle begins. Typically, the off-time cycle is a 30-day countdown, however, is adjustable. Additionally, electrical connection 414 provides a power supply to injection pump 412.

As previously stated, injection pump 412 is disposed within containment tank 416. Injection pump 412 may be drilled, epoxied, strapped, or connected to the side, bottom, or anywhere within containment tank 416. Injection pump 412 may have a wide range in power output from 1/125 HP to 7.5 HP depending on its specific application. Injection pump 412 utilizes a component such as an impeller, vane, piston, or any suitable dynamic component which produces a drop in pressure towards flow line 418. Subsequently, chemical treatment within containment tank 416 travels into flow line 418. Flow line 418 may be any hollow material rigid capable of transporting algaecide.

Information processed by the microcontroller 408 may come from at least one level switch 424 disposed outside or inside containment tank 416 via electrical connection 420. In FIG. 4, level switch is disposed inside containment tank 416, but level witch 424 may be disposed outside containment tank 416 as well. The purpose of each level switch 424 is to track the volume of chemical treatment in containment tank 416 and transmit a warning signal via electrical connection 420 when the chemical treatment within containment tank 416 lowers beyond a pre-determined threshold. Each level switch 424 may use a mercury switch inside a hinged float, raise a rod to actuate a microswitch, or use a reed switch mounted in a tube; a float, containing a magnet, surrounds the tube and is guided by it. Optionally, any known employment of at least one sensor may be used to track the volume of containment tank 416 and transmit warning signals to microcontroller 408 via electrical connection 420.

Flow meter 410 may be a turbine meter comprised of a magnet which triggers a Hall Effect transistor. A momentary pulse from the Hall Effect transmits flow volume measurements via electrical connection 420 to the microcontroller 408. Monetary pulses are computed to form the actual flow volume within the microcontroller 408 and measured against the assigned flow volume. Assigned flow volume may range per the application of the A/C unit but in standard applications is .3 liters. A turbine meter is provided in this embodiment, however any suitable manner to calculate the flow volume may be implemented.

Control panel 406 provides internal wiring, power, and support for microcontroller 408. In addition to microcontroller 408, disposed on control panel 406 is low level light 428, low flow light 430, pump running light 432, horn 434, system reset push button 436, prime push button 438, on/off switch 444, system start push button 446 and power on light 442. All of which are powered and connected to microcontroller 408 via internal wiring. While sufficient power is supplied to ACID 400 when the on/off switch is flipped “on”, microcontroller 408 will transmit a signal via electrical connection 404 illuminating power on light 442. However, when the on/off switch is flipped “off”, ACID 400 will power down and no processing will occur in microcontroller 408.

The information provided from the level switches 424 may allow microcontroller 408 to accurately compute a volume of containment tank 416. Once volume of containment tank 416 drops below a threshold, microcontroller 408 may transmit control signals to low level light 428 and horn 434 via internal wiring within controller 406. Microcontroller 408 signals horn 434 to activate and illuminate low level light 428, identifying a “low volume fault”. Additionally, the microcontroller 408 may transmit a yield signal to injection pump 412 to lock via electrical connection 414. The sounding of horn 434 may notify homeowner to check the status of ACID 400. Additionally, the homeowner may turn off the horn by pressing silence alarm silence push button 440 located on control panel 406 prior to refilling containment tank 416. Once containment tank 416 is refilled and exceeds the threshold of volume within containment tank 416, system reset push button 436 located on control panel 406 is pressed. However, if the button is not pressed microcontroller 408 transmits a control signal to turn off low level light 428 and unlock injection pump 416. In either case ACID 400 undergoes a hard reset turning off low level light 428 and deactivating horn 434 if not already manually turned off via silence alarm silence push button 440. Then, system start button 446 is pressed subsequently, injection pump 412 pumps the assigned flow volume into flow line 418 and the off-time cycle resets. In an alternative embodiment, microcontroller 408 may not transmit a yield signal to lock injection pump 412 when a “low volume fault” occurs. Rather microcontroller 408 may only transmit control signals to low level light 428 and horn 434 via internal wiring within controller 406. As previously stated, the homeowner may deactivate horn 434 by pressing silence alarm silence push button 440 located on control panel 406 prior to refilling containment tank 416. Additionally, once containment tank 416 is refilled and exceeds the threshold of volume within containment tank 416, system reset push button 436 located on control panel 406 is pressed. However, if the button is not pressed microcontroller 408 transmits a control signal to turn off low level light 428 and the off-time cycle resets. Then, system start button 446 is pressed subsequently, injection pump 412 pumps the assigned flow volume into flow line 418 and the off-time cycle resets.

Additionally, Microcontroller 408 receives information from flow meter 410 via electrical connection 420 as a measurement of flow volume. The flow volume measurement is input into a flow algorithm which calculates the actual flow volume of chemical treatment through flow line 418. If the actual flow volume is less than flow volume threshold of the assigned flow, the microcontroller 408 may transmit a yield signal to injection pump 412 to lock the pump, a control signal to light low flow light 430, and/or a control signal to activate horn 434. Similarly, Flow volume threshold of the assigned flow may range, however is typically 50% of the actual flow to assigned flow.

Similar to the low volume case, horn 434 will prompt the homeowner to check the status of ACID 400. Upon inspection, the homeowner may optionally press the alarm silence push button 440 (deactivate horn 434) and inspect that the low flow light 430 illuminated identifying a “low flow fault.” Low flow light 430 is indicative of injection pump 412 failing to pump assigned flow volume to flow line 418 or an issue with flow line 418. An issue with flow line 418 may be a tight connection, leak, or any failure of the transportation of algaecide. Additionally, any number of multiple flow meters 410 may be installed into flow line 418 at any location inside or outside containment tank 416 (not illustrated). Each flow meter 410 is connected to microcontroller 408 via electrical connection 420. Thus, the actual flow volume at any stage in flow line 418 from containment tank 416 to solenoid valve 422 may be measured by a flow meter 410 of any application and transmitted to microcontroller 408 via electrical connection 420.

If the actual flow volume is measured to be less than the flow volume threshold of the assigned flow at any flow meter 410 along the flow line path 418, as previously stated horn 434 will activate and low flow light 430 will be illuminated identifying a “low flow fault”. As a result of the low flow, the homeowner may contact the manufacture, any certified repairer, or fix the issue “in house.” Low flow is an unexpected and rare fault, requiring more maintenance than refilling containment tank 416. Thus, extra maintenance such as repairing or replacing injection pump 416 may require ordering parts or a whole replacement of injection pump 416. However, the integrity of injection pump 416 may be intact, yielding a leak or clog along flow line 418. In the event of a clog or leak, flow line 418 is to be inspected. A leak will be apparent of spilled chemical treatment requiring sealing the leak with epoxy or any type of know sealant. However, there may if there is no leak flow line 418 may be disassembled and inspected for internal debris or tight connections between adjacent segments in the flow line 418 forcing a clog. Once injection pump 416 is repaired or replaced or flow line 418 is cleared or sealed, the reset system push button 436 is pressed. ACID 400 undergoes a hard reset turning off low flow light 430 and deactivating horn 434 if not already manually turned off via alarm silence push button 440. Then, system start button 446 is pressed subsequently, injection pump 412 pumps the assigned flow volume into flow line 418 and the off-time cycle resets.

Prime push button 438 provides support for maintenance. When pressed push button 438 transmits a signal to microcontroller via electrical connection 404. Microcontroller 408 will initiate injection pump 412 to pump continuously until prime push button 438 is released. This in turn allows a homeowner or repair man to better evaluate and diagnose a “low flow fault”.

In the event in which a “low volume fault” occurs and the horn 434 is deactivated, but no yield signal is sent from microcontroller 408 to injection pump 412, injection pump 412 will continue to pump. Eventually a “low flow fault” will occur when there is no more chemical treatment left in containment tank 416. The same procedure as described above to resolve a “low flow fault” alleviates the fault.

Under normal circumstances ACID 400 runs with neither the “low flow fault” or “low volume fault” and injects chemical treatment into effluent drain line 112 consistently. After the off-time cycle of the timer within microcontroller 408 ends, microcontroller 408 transmits a control signal to injection pump 412 via electrical connection 414. Simultaneously, microcontroller 408 transmits a control signal illuminating pump running light 432 via internal connections within control panel 406. The control signal initiating injection pump 412 comprises the assigned flow volume. Thus, the assigned flow volume may be programmable within microcontroller 408. Additionally, flow volume threshold of the assigned flow and the threshold of volume within containment tank 416 may be programmable as well.

In embodiments, microcontroller 408 may be connected to the homeowner's internet via internet connection 426. Internet connection 426 may be wireless or an ethernet connection. Internet access allows homeowners a user interface compatible with microcontroller 408. The user interface may be an application on their phone, a browser, or any medium allowing the homeowner to communicate with microcontroller 408 by receiving information from microcontroller 408 or giving microcontroller 408 a command. Features of the user interface may be displaying data from microcontroller 408 such as the current volume of the containment tank 416, previous flow rates through the flow line 418, or checking what the fault is upon hearing horn 434. Optionally, the user interface may notify the homeowner of a fault in any suitable method including but not limited to a text message, email, or push notification through the application. Additional features may include command functions such as remotely deactivating horn 434, ending the off-time cycle prematurely and autonomously activating ACID, or restarting a command which effectively presses system reset push button 436.

In other embodiments, features may be implemented if multiple flow meters 410 may be installed along flow line 418. For example, in the event of a “low flow fault” measurements from multiple flow meters may identify if the injection pump 412 is fully operational or if there is a leak or clog in the flow line 418 and if so, where the leak or clog is. Thus, information for repairing a “low flow fault” may be provided to make the repair more efficient and faster. Different seasons of the year require different frequencies of A/C use for the individual homeowners. Further, the user interface may allow the homeowner to program the off-time cycle to be extended or shortened and to increase or decrease flow volume. The user interface will record a digital catalog of each injection unbounding the homeowner of the responsibility of keeping track of chemical treatment injections manually.

Multiple shapes and capacities of containment tank 416 may be utilized depending on its application and placement within the house. For example, the containment tank may be round and cylindrical or rigid boxed and may range in non-limiting capacities of 0.5 to 100 gallons. In some embodiments, ACID 400 may be located inside air handling unit 106 (e.g., referring to FIG. 1) adjacent to effluent drain line 112. However, the location of ACID 400 is not limited to inside or proximity of the air handling unit 106. Alternatively, ACID 400 may be located anywhere in structure 102 or outside 104 (e.g., referring to FIG. 1). For example, ACID 400 may be located in the homeowner's garage or utility closet wherein containment tank 416 and injection pump 412 are larger than if contained in air handling unit 106. In summary, ACID 400 may be configured to meet the needs of clearing an effluent line 112 in the application of any air conditioning system 100 (e.g., referring to FIG. 1). Further, any implementation of power or information transfer in the disclosure may be supported by wireless implementation as well as direct connections.

Current technology does not include the systems and methods for autonomously injecting chemical treatment into an effluent drain line 112 (e.g., referring to FIG. 1) as discussed above. Specifically, current technology has not been adapted to provide a system or method to autonomously eliminate blockages 300 (e.g., referring to FIG. 3) in effluent drain lines 112. The disclosure provides improvements to known methods and systems which adds value and utility to the homeowner. For example, to prevent blockages 300 of effluent drain line 112 a homeowner needs to manually access the effluent drain line 112 routinely to inject chemical treatment and record each injection. However, this is a tedious chore and commonly forgotten. If left untreated, effluent drain lines 112 may clog due to blockage 300, water will back up into condensation tray 214 (e.g., referring to FIG. 1) and potentially overflow into structure 102 (e.g., referring to FIG. 1), potentially causing damage and costly repairs to structure 102. Range of repairs might include mobilizing an A/C Technician to unclog blockage 300 in effluent drain line 112 and restart the A/C system or as costly as major drywall, carpet, or home repair due to water leakage.

Additionally, it may be dangerous to access to effluent drain line 112 in hot attic spaces or where air handling unit 106 (e.g., referring to FIG. 1) is located. There may have poorly light and cramped spaces. A homeowner may have to climb narrow stairs haul dangerous chemicals and maneuver an unfamiliar environment. Methods and systems described above for ACID 400 limits the homeowner's frequency to access these areas or eliminates it all together.

The methods/systems/compositions/tools may include any of the various features disclosed herein, including one or more of the following statements.

Statement 1: A system comprising a containment tank, an injection pump disposed within the containment tank, at least one level switch disposed on or inside the containment tank, a flow line disposed at least partially within the containment tank, at least one flow meter disposed along the flow line, and a microcontroller. The microcontroller is configured to: maintain an off-time cycle, transmit a signal to the injection pump to pump a volume of chemical treatment stored in the containment tank into the flow line after the off-time cycle ends, track the volume of chemical treatment passing through the flow line with the at least one flow meter to produce a flow rate, inject the volume of chemical treatment from the flow line into an effluent drain line, track a current volume of the containment tank with the at least one level, and restart the off-time cycle.

Statement 2: The system of Statement 1, wherein the microcontroller is further configured to activate a horn and illuminate a first fault light when the volume of chemical treatment within containment tank reaches below a threshold based on the current volume of the containment tank.

Statement 3: The system of Statement 1, wherein the microcontroller is further configured to activate a horn and illuminate a second fault light when the volume of chemical treatment passing through the flow line drops below a threshold based on the flow rate.

Statement 4: The system of Statement 1, wherein the microcontroller is further configured to activate and deactivate a horn.

Statement 5: The system of Statement 1, wherein the flow line is configured to transport the volume of chemical treatment from the containment tank to an effluent line via pressure from the injection pump.

Statement 6: The system of Statement 1, wherein the at least one flow meter is placed on the flow line between the containment tank and an effluent line.

Statement 7: The system of Statement 1, wherein multiple solenoid valves are placed at an end of the flow line and inject the volume of chemical treatment into separate effluent lines.

Statement 8: The system of Statement 4, wherein the microcontroller is connected to a user interface and the user interface receives inputs to control the microcontroller.

Statement 9: The system of Statement 8, wherein the user interface is configured to: deactivate the horn, reset the off-time cycle and run the injection pump, and run the injection pump without resetting the off-time cycle.

Statement 10: The system of Statement 8, wherein the user interface may provide the current volume of the containment tank, previous flow rates, or a distinction of fault 1 or fault 2.

Statement 11: A method comprising: maintaining an off-time cycle with a microcontroller configured to control an injection of a volume of chemical treatment into an effluent drain line, pumping the volume of chemical treatment stored in a containment tank into a flow line after the off-time cycle ends with an injection pump, tracking the volume of chemical treatment passing through the flow line with at least one flow meter to produce a flow rate, injecting the volume of chemical treatment from the flow line into an effluent drain line, tracking a current volume of the containment tank with at least one level switch, and restarting the off-time cycle.

Statement 12: The method of Statement 11, further comprising activating a horn and illuminating a first fault light when the volume of chemical treatment within containment tank reaches below a threshold based on the current volume of the containment tank.

Statement 13: The method of Statement 11, further comprising activating a horn and illuminating a second fault light when the volume of chemical treatment passing through the flow line drops below a threshold based on the flow rate..

Statement 14: The method of Statement 11, further comprising deactivating a horn if it has already been activated.

Statement 15: The method of Statement 11, wherein the flow line is configured to transport chemical treatment from the containment tank to an effluent line via pressure from the injection pump.

Statement 16: The method of Statement 11 wherein the at least one flow meter is placed on the flow line between the containment tank and an effluent line.

Statement 17: The method of Statement 11 wherein multiple solenoid valves are placed at an end of the flow line and inject chemical treatment into separate effluent lines.

Statement 18: The method of Statement 14 further comprising connecting the microcontroller to a user interface via a wireless connection.

Statement 19: The method of Statement 18 further comprising: deactivating the horn with the user interface, deactivating an alarm indication with the user interface, and running the injection pump without resetting the off-time cycle with the user interface.

Statement 20: The method of Statement 18 wherein the user interface may provide the current volume of the containment tank, previous flow rates, or a distinction of fault 1 or fault 2.

The foregoing figures and discussion are not intended to include all features of the present techniques to accommodate a buyer or seller, or to describe the system, nor is such figures and discussion limiting but exemplary and in the spirit of the present techniques.

What is claimed is:

1. A system comprising:
a containment tank;
an injection pump disposed within the containment tank;
at least one level switch disposed on or inside the containment tank;
a flow line disposed at least partially within the containment tank;
at least one flow meter disposed along the flow line; and
a microcontroller configured to:
maintain an off-time cycle;
transmit a signal to the injection pump to pump a volume of chemical treatment stored in the containment tank into the flow line after the off-time cycle ends;
track the volume of chemical treatment passing through the flow line with the at least one flow meter to produce a flow rate;
inject the volume of chemical treatment from the flow line into an effluent drain line;
track a current volume of the containment tank with the at least one level; and
restart the off-time cycle.

2. The system of claim 1, wherein the microcontroller is further configured to activate a horn and illuminate a first fault light when the volume of chemical treatment within containment tank reaches below a threshold based on the current volume of the containment tank.

3. The system of claim 1, wherein the microcontroller is further configured to activate a horn and illuminate a second fault light when the volume of chemical treatment passing through the flow line drops below a threshold based on the flow rate.

4. The system of claim 1, wherein the microcontroller is further configured to activate and deactivate a horn.

5. The system of claim 1, wherein the flow line is configured to transport the volume of chemical treatment from the containment tank to an effluent line via pressure from the injection pump.

6. The system of claim 1, wherein the at least one flow meter is placed on the flow line between the containment tank and an effluent line.

7. The system of claim 1, wherein multiple solenoid valves are placed at an end of the flow line and inject the volume of chemical treatment into separate effluent lines.

8. The system of claim 4, wherein the microcontroller is connected to a user interface and the user interface receives inputs to control the microcontroller.

9. The system of claim 8, wherein the user interface is configured to:
deactivate the horn;
reset the off-time cycle and run the injection pump; and
run the injection pump without resetting the off-time cycle.

10. The system of claim 8, wherein the user interface may provide the current volume of the containment tank, previous flow rates, or a distinction of fault 1 or fault 2.

11. A method comprising:
maintaining an off-time cycle with a microcontroller configured to control an injection of a volume of chemical treatment into an effluent drain line;
pumping the volume of chemical treatment stored in a containment tank into a flow line after the off-time cycle ends with an injection pump;
tracking the volume of chemical treatment passing through the flow line with at least one flow meter to produce a flow rate;
injecting the volume of chemical treatment from the flow line into an effluent drain line;
tracking a current volume of the containment tank with at least one level switch; and
restarting the off-time cycle.

12. The method of claim 11, further comprising activating a horn and illuminating a first fault light when the volume of chemical treatment within containment tank reaches below a threshold based on the current volume of the containment tank.

13. The method of claim 11, further comprising activating a horn and illuminating a second fault light when the volume of chemical treatment passing through the flow line drops below a threshold based on the flow rate.

14. The method of claim 11, further comprising deactivating a horn if it has already been activated.

15. The method of claim 11, wherein the flow line is configured to transport chemical treatment from the containment tank to an effluent line via pressure from the injection pump.

16. The method of claim 11, wherein the at least one flow meter is placed on the flow line between the containment tank and an effluent line.

17. The method of claim 11, wherein multiple solenoid valves are placed at an end of the flow line and inject chemical treatment into separate effluent lines.

18. The method of claim 14, further comprising connecting the microcontroller to a user interface via a wireless connection.

19. The method of claim 18, further comprising:
deactivating the horn with the user interface;
deactivating an alarm indication with the user interface; and
running the injection pump without resetting the off-time cycle with the user interface.

20. The method of claim 18, wherein the user interface may provide the current volume of the containment tank, previous flow rates, or a distinction of fault 1 or fault 2.
